(19) United States
(12) Patent Application Publication
(10) Pub. No.: US 2025/0000637 A1
(43) Pub. Date: Jan. 2, 2025

(54) SURGICAL TOOLS, SYSTEMS, AND METHODS

(71) Applicant: Arthrosurface, Inc., Franklin, MA (US)

(72) Inventors: Nam CHAO, Bedford, MA (US); Steven W. EK, Durham, NH (US)

(21) Appl. No.: 18/760,716

(22) Filed: Jul. 1, 2024

Related U.S. Application Data
(60) Provisional application No. 63/511,530, filed on Jun. 30, 2023.

(51) Int. Cl. A61F 2/08

(52) U.S. Cl. CPC A61F 2/0805; A61F 2002/0829; A61F 2002/0858; A61F 2002/0888; A61F 2220/0016

(57) ABSTRACT

This disclosure includes apparatuses for simultaneous delivery of a fastener and an implant; systems including at least one of the present apparatuses, a seal assembly, and a cannula; caddy systems configured to receive a fastener and/or an implant for use with the present apparatuses; kits including at least one of the present apparatuses or systems and at least one of the present caddies; and methods of simultaneously delivering a fastener and an implant with the present apparatuses. The present apparatuses include an elongated body, one or more spikes at a body distal end of the body, a hub configured to receive a proximal portion of the body, and a handle coupled to the hub. The body defining a body recess in a peripheral surface of the body at point that is offset from the body distal end, and a body passage extending between a body proximal end and a body distal opening within the body recess. The apparatus further includes an elongated pusher and a slider coupled to a proximal end of the pusher, the slider configured to move longitudinally relative to the handle from a retracted position to a deployed position to move the pusher distal end longitudinally away from the body distal opening and laterally outward from the body.

### CROSS-REFERENCE TO RELATED APPLICATIONS

This application claims the benefit of U.S. Provisional Patent Application No. 63/511,530, filed on Jun. 30, 2023, which is fully incorporated herein by reference.

### FIELD OF INVENTION

The present invention relates generally to surgical fasteners, more particularly, but not by way of limitation, to fasteners, tools, and methods for surgical repair of anatomical structures, such as tendons (e.g., the supraspinatus tendon, commonly known as the rotator cuff).

### DESCRIPTION OF RELATED ART

Various surgical fasteners and delivery tools are known, such as for securing an implant, for example for repair of a tear in the supraspinatus tendon (which may be commonly referred to as a rotator cuff tear). For example, United States Patent Application No. US 2008/0188936 discloses certain examples of fasteners and implants for such repairs.

### SUMMARY

This disclosure includes various apparatuses, kits, systems, and methods for simultaneous delivery of a fastener and an implant.

In some configurations of the present apparatuses for simultaneous delivery of a fastener and an implant, the apparatus comprises: an elongated body, one or more spikes, a hub, a handle, an elongated pusher, and a slider. In some such configurations, the elongated body extends from a body proximal end to a body distal end, the body defines a body proximal opening through the body proximal end, a body recess in a peripheral surface of the body at point that is offset from the body distal end, and a body passage extending between the body proximal opening and a body distal opening within the body recess. In some such configurations, the one or more spikes each have a spike proximal end coupled to the body distal end, and a spike distal end extending from the body distal end, and the spike proximal end has a transverse dimension that is smaller than a transverse dimension of the body distal end such that a shoulder is defined at the spike proximal end, where the shoulder is configured to abut a head of a fastener received over the spike. In some such configurations, the hub has a hub proximal end and a hub distal end, and the hub configured to receive the proximal end of the body through the hub distal end, and the handle is coupled to the hub proximal end. In some such configurations, the elongated pusher has a pusher proximal end and a pusher distal end, and the pusher is disposed in the body passage such that the pusher distal end is disposed beyond the body distal opening. In some such configurations, the slider is coupled to the pusher proximal end, and is configured to move longitudinally relative to the handle from a retracted position to a deployed position to move the pusher distal end longitudinally away from the body distal opening and laterally outward from the body.

In some configurations of the present apparatuses, the one or more spikes comprise a pair of spikes, where each of the pair of spikes are parallel to and laterally spaced from the other of the pair of spikes.

In some configurations of the present apparatuses, the hub is coupled in fixed relation to the body, and the handle is rotatable relative to the hub to cause the pusher distal end to rotate relative to the body. In some such configurations, apparatus is configured such that moving the slider longitudinally relative to the handle, or rotating the handle relative to the hub, will move the distal end of the pusher independent of the body distal end.

In some configurations of the present apparatuses, the body recess defines a sloped surface spaced from the body distal opening, and where the apparatus is configured such that, when the slider is moved from the retracted position to the deployed position, a flexible portion of the pusher contacts the sloped surface to cause the pusher distal end to extend laterally outward from the body.

In some configurations of the present apparatuses, the pusher comprises a roller coupled to the pusher distal end.

Some configurations of the present apparatuses further comprise: a seal assembly comprising: an annular seal body and a resilient membrane. In some such configurations, the annular seal body having a seal proximal end and a seal distal end, the seal body defines a seal passage extending through and between the seal proximal end and the seal distal end, and a tapered distal surface disposed around the seal passage at the seal distal end. In some such configurations, the resilient membrane is disposed at least around an interior periphery of the seal passage, and is configured to cooperate with the body of the apparatus to substantially seal the seal passage when the body of the apparatus extends through the resilient membrane and the seal passage. In some such configurations, the elongated body of the apparatus is disposed through the resilient membrane of the seal and the seal passage with the seal distal end facing the body distal end.

In some configurations of the present systems, the system comprises: one or more of the present apparatuses comprising a seal assembly, and a cannula. In some such configurations, the cannula comprises a cannula distal end, a cannula proximal end configured to be coupled to the seal body, and a cannula passage extending through and between the cannula proximal end and the cannula distal end, the cannula further defining a cannula proximal surface tapered distally and inward toward the cannula passage, the cannula proximal surface configured to cooperate with the seal distal surface to provide a seal between the cannula proximal surface and the seal distal surface. In some such configurations, an outer surface of the cannula includes one or more threads that are configured to engage soft tissue as the cannula is rotated.

In some configurations of the present systems, a proximal end of the cannula includes an enlarged portion that is configured to be engaged by a user to vary the depth and/or direction of the cannula.

In some configurations of the present systems, the cannula distal end defines a notch configured to permit a portion of the pusher to deflect radially outward through the notch.

Some configurations of the present systems further comprise: an obturator having an elongated body with a obturator proximal end and a tapered obturator distal end, the obturator body configured to extend through the cannula such that the obturator distal end extends distally beyond the cannula distal end to facilitate insertion of the cannula through soft tissue of a patient.

In some configurations of the present systems, the system comprises: a guide cannula having a slide portion and a handle portion. In some embodiments, the slide portion is configured to be at least partially disposed in the cannula passageway. In some embodiments, the slide portion has an outer surface having a convex curvature and an inner surface having a convex curvature the inner surface extending between a first edge second edge of the slide portion, wherein the inner surface forms a sector having a central angle between 160-180 or 180-200 degrees. In some embodiments, the central angle is 180 degrees. In some embodiments, the slide portion extends from a distal end to approximal end, wherein the slide portion comprises a tapered region extending from the distal end to a distance spaced apart from the proximal end. In some embodiments, the slide portion has an outer dimension smaller than the dimensions of the cannula passageway and an inner surface having a dimension larger than the outer dimension of a distal end of the apparatus, such that the cannula guide protects the cannula from being punctured by the one or more spikes of the apparatus.

In some configurations of the present caddy systems, the caddy system comprises: a base portion defining one or more first fastener recesses; and a lid portion coupled to the base portion and configured to be rotated relative to the base portion to selectively cover or expose the first fastener recess(es). In some embodiments, a recess is disposed at least partially in the lid portion or at least partially in the base portion, wherein the recess is configured to receive an implant.

In some configurations of the present caddy systems, the base portion further defines one or more implant recesses each overlapping at least one of the one or more first fastener recesses, where the lid portion is configured to be rotated relative to the base portion to selectively cover or expose the implant. In some such configurations, the one or more implant recesses includes a substantially planar lower surface offset below a substantially planar upper surface of the base.

Some configurations of the present caddy systems further comprise: a flexible, fibrous implant disposed over at least one of the first fastener recesses; and a first fastener extending through the implant and into the at least one of the first fastener recesses; where the lid portion is rotatable, relative to the base, over the first fastener and at least a portion of the implant.

In some configurations of the present caddy systems, the body further defines one or more second fastener recesses, where the lid portion is configured to be rotated relative to the base portion to selectively cover or expose the second fastener recess(es). Some such configurations further comprise: a second fastener extending into at least one of the second fastener recesses; where the lid portion is rotatable, relative to the base, over the second fastener. In some embodiments, a recess is disposed at least partially in the lid portion or at least partially in the base portion, wherein the recess is configured to receive an implant

In some implementations of the present methods, the method comprises: disposing a shield distal end of one of the present apparatuses apparatus adjacent a bone of a patient, where the slider is in the retracted position, where a fastener is disposed over the one or more spikes, and the fastener extends through a flexible, fibrous implant; inserting the fastener into the bone to secure a first portion of the implant to the bone; and moving the slider from the retracted position toward the deployed position to urge a second portion of the implant away from the body of the apparatus and toward a soft tissue structure of the patient.

In other implementations of the present methods, the method comprises: inserting the elongated body of one of the present apparatuses through a cannula that is disposed through soft tissue of a patient, the cannula comprising a cannula distal end, a cannula proximal end configured to be coupled to the seal body, and a cannula passage extending through and between the cannula proximal end and the cannula distal end, the cannula further defining a cannula proximal surface tapered distally and inward toward the cannula passage, the cannula proximal surface configured to cooperate with the seal distal surface to provide a seal between the cannula proximal surface and the seal distal surface. In some such implementations, during insertion, a seal assembly is disposed around the elongated body of the apparatus, the seal assembly comprising: an annular seal body having a seal proximal end and a seal distal end, the seal body defining a seal passage extending through and between the seal proximal end and the seal distal end, and a tapered distal surface disposed around the seal passage at the seal distal end; and a resilient membrane disposed at least around an interior periphery of the seal passage, the resilient membrane configured to cooperate with the body of the apparatus to substantially seal the seal passage when the body of the apparatus extends through the resilient membrane and the seal passage. Some such implementations further comprise: coupling the seal body to the proximal end of the cannula such that the seal distal surface cooperates with the cannula proximal surface to provide a seal between the cannula proximal surface and the seal distal surface. Some such implementations further comprise: after moving the slider from the retracted position toward the deployed position, rotating the handle relative to the hub to urge one or both of two lateral edges of the second portion of the implant toward the soft tissue structure. Some such implementations further comprise: inserting a fastener through the second portion of the implant and into the soft tissue structure to couple the second portion of the implant to the soft tissue structure.

In other implementations of the present methods, the method comprises: inserting the elongated body of one of the present apparatuses through the resilient membrane and the seal passage of one of the present seals such that the seal distal end faces the body distal end. Some such implementations further comprise: inserting, while the elongated body is disposed through the resilient membrane and seal passage, a fastener disposed over the one or more spikes through a flexible, fibrous implant. Some such implementations further comprise: inserting the spikes of the apparatus through a fastener that is disposed through a flexible, fibrous implant.

In some implementations of the present methods, the method comprises: disposing a slide portion of a cannula guide at least partially in the cannula passage such that the slide portion at least partially encircles a portion of the apparatus within the cannula passage. In some embodiments, the slide portion has an outer surface having a convex curvature and an inner surface having a convex curvature the inner surface extending between a first edge second edge of the slide portion, wherein the inner surface forms a sector having a central angle between 160-180 or 180-200 degrees. In some embodiments, the central angle is 180 degrees. In some embodiments the slide portion has an outer dimension smaller than a dimensions of the cannula passageway and an inner surface having a dimension larger than the outer dimension of a distal end of the apparatus, such that the cannula guide protects the cannula from being punctured by the one or more spikes of the apparatus.

In some configurations of the present kits, the kit comprises: one or more of the present apparatuses and/or one or more of the present systems; and one or more of the present caddy systems.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device or system that is configured in a certain way is configured in at least that way, but it can also be configured in other ways than those specifically described.

Any embodiment of any of the present apparatuses and methods can consist of or consist essentially of-rather than comprise/include/contain/have-any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

As used herein, the term “caddy” refers to a storage container having one or more divisions. For example, a caddy may be a storage container having a first region adapted to retain one or more bone staples and a second region adapted to retain one or more bone tacks.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 is a perspective view of a first embodiment of the present systems, including a cannula, an obturator, a seal assembly, and a delivery tool in a first configuration.

FIG. 2 is the cannula and the obturator of FIG. 1.

FIG. 3 is a proximal perspective view of the cannula of FIG. 1.

FIG. 4A is a proximal perspective view of the seal assembly of FIG. 1.

FIG. 4B is a distal perspective view of the seal assembly of FIG. 1.

FIG. 5A is a perspective view of the delivery tool and seal assembly of FIG. 1, with the delivery tool in the a configuration.

FIG. 5B is a distal end view of the delivery tool and seal assembly of FIG. 5A.

FIG. 5C is a side cross-sectional view of the delivery tool and seal assembly of FIG. 5A.

FIG. 6 is a partially exploded, cross-sectional side view of the delivery tool of FIG. 1.

FIG. 7 is a side cross-sectional view of the delivery tool of FIGS. 5A-5C, shown in a second configuration.

FIGS. 8A and 8B are upper and lower perspective views, respectively, of a first embodiment of the present caddy systems shown pre-loaded with implants and fasteners.

FIG. 9 is a top view of a base portion of the caddy system of FIGS. 6A-6B.

FIG. 10A is a top view of the caddy system of FIGS. 8A-8B with a lid portion of the caddy system in a first configuration relative to the base.

FIG. 10B is a side cross-sectional view of the caddy system of FIG. 10A.

FIG. 11A is a top view of the caddy system of FIGS. 8A-8B with a lid portion of the caddy system in a second configuration relative to the base.

FIG. 11B is an exploded, side cross-sectional view of the caddy system of FIG. 11A.

FIG. 12 is a perspective view of the delivery tool of FIG. 1 engaging a fastener of the caddy system of FIGS. 8A-8B.

FIG. 13 is a perspective view of the delivery tool of FIG. 1 deploying a fastener to a bone of a patient to secure a first portion of an implant relative to the bone.

FIG. 14 is a perspective view of the delivery tool of FIG. 1, in the second configuration, deploying a second portion of the implant toward soft tissue.

FIGS. 15A-15H depict perspective views of various configurations of a distal end of a pusher of the delivery tool.

FIG. 16A is a sequence of side views of a distal end of the delivery tool with a further embodiment of the pusher, illustrating the deployment of an implant.

FIG. 16B is a side view of a distal end of the delivery tool with a further embodiment of the pusher.

FIG. 17 is a perspective view of a further embodiment of the obturator of FIGS. 1 and 2.

FIG. 18 is a distal perspective view of a further embodiment of the seal assembly of FIGS. 1 and 4A-4B.

FIG. 19 is a distal perspective view of a further embodiment of the seal assembly of FIGS. 1, 4A-4B, and 16.

FIG. 20 is a distal perspective view of a further embodiment of the cannula of FIGS. 1, 2, and 3.

FIG. 21 is a distal perspective view of a further embodiment of the cannula of FIGS. 1-3 and 18.

FIGS. 22A and 22B are upper perspective views of a further embodiment of the caddy system of FIGS. 8A-11B.

FIGS. 23A and 23B are upper perspective views of a further embodiment of the caddy system of FIGS. 22A and 22B.

FIGS. 24A, 24B, and 24C illustrates a side, perspective, and distal view of a cannula guide, according to embodiments described herein.

### DESCRIPTION OF ILLUSTRATIVE EMBODIMENTS

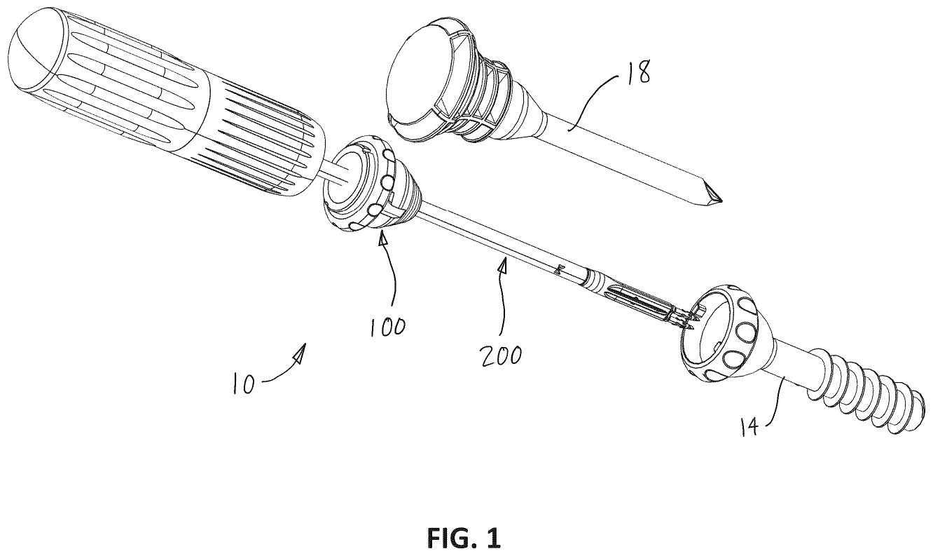

Referring now to the drawings, and more particularly to FIG. 1, shown there and designated by the reference numeral 10 is a first embodiment of the present systems. System 10 includes a cannula 14, an obturator 18, a seal assembly 100, and a delivery tool 200. As shown, seal 100 is configured to receive a portion of tool 200 such that the tool can be inserted into the cannula. As described in more detail below, seal assembly 200 is configured to seal around the portion of the tool in the seal assembly, as well as to be coupled to a proximal end of cannula 14 to form a seal between seal assembly 100 and cannula 14.

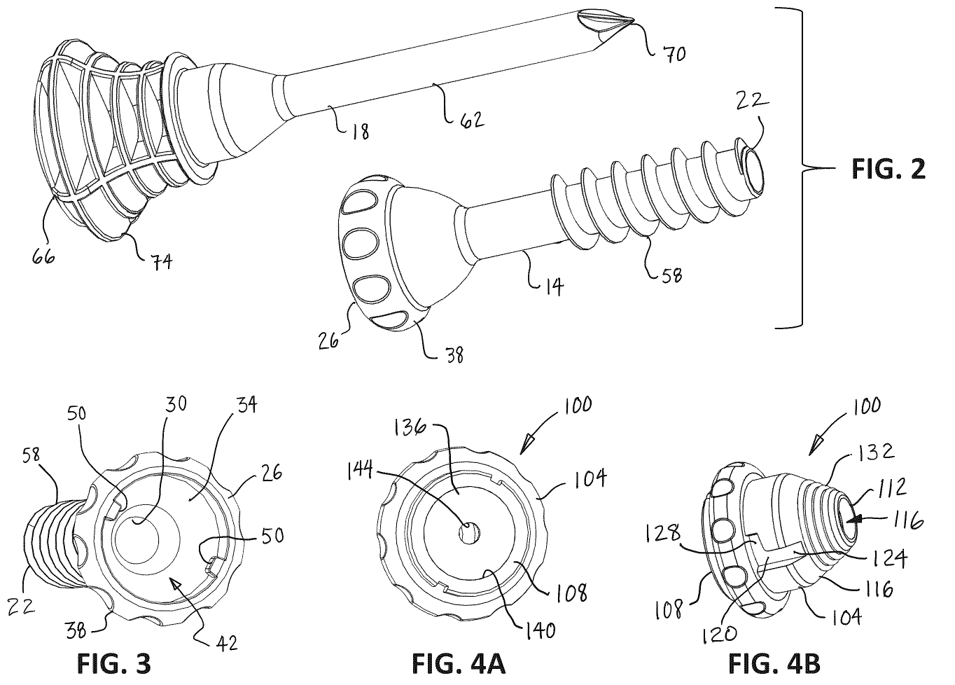

Referring now to FIGS. 2-3, FIG. 2 shows an enlarged perspective view of cannula 14 and obturator 18, and FIG. 3 shows a proximal perspective view of cannula 14 alone. As shown, cannula 14 comprises a cannula distal end 22, a cannula proximal end 26 configured to be coupled to the seal assembly (100), and a cannula passage 30 extending through and between the cannula proximal end and the cannula distal end. As shown in FIG. 3, cannula 14 also defines a cannula proximal surface 34 tapered distally (toward cannula distal end 22) and inward toward the cannula passage (30). Cannula proximal surface 34 is configured to cooperate with a distal surface of seal assembly 100 to provide a seal therebetween. In the embodiment shown, at cannula proximal end 26 is a head or hub 38 that is enlarged in the radial direction relative to at least longitudinally adjacent portions of the cannula. Head or hub 38 is configured to be grasped by a user to vary the depth and/or direction of the cannula, such as when the cannula is inserted through soft tissue of a patient. In this configuration, cannula 14 defines a recess 42 that extends from cannula proximal end 26 inward toward cannula distal end 22, with cannula proximal surface 34 disposed at the bottom or distal-most portion of recess 42. Cannula 14 can also be configured to engage seal assembly 100, such as by threads, projections and corresponding grooves, an interference fit, or the like. For example, and as best seen in FIG. 3, cannula 14 defines two projections 46 that extend inward from the inner peripheral surface 50 that defines recess 42. As described in more detail below, projections 46 are configured to engage seal assembly 100 to resist separation of the cannula and the seal assembly. In the depicted embodiment, an outer surface 54 of the cannula includes one or more threads 58 that are configured to engage soft tissue of a patient as the cannula is rotated.

Obturator 18 has an elongated body 62 with a obturator proximal end 66 and an tapered obturator distal end 70. As shown, obturator 18 includes an enlarged head or handle region 74 at obturator proximal end 66. In use, body 62 is configured to extend through cannula 14 such that obturator distal end 70 extends distally beyond cannula distal end 22 to facilitate insertion of the cannula through soft tissue of a patient. For example, a user such as a physician can make an initial incision through the skin of a patient, and then the obturator and cannula can be together inserted through the incision. More specifically, distal end 70 is first inserted through the incision such that the tapered shape expands the incision to enable the user to rotate cannula 14 and thereby cause threads 58 to engage and draw the cannula into the tissue (and/or draw the tissue outward over the threads).

Referring now to FIGS. 4A and 4B, FIG. 4A shows a proximal perspective view of seal assembly 100, and FIG. 4B shows a distal perspective view of seal assembly 100. As shown, seal assembly 100 comprises: an annular seal body 104 having a seal proximal end 108 and a seal distal end 112. Seal body defines a seal passage 116 extending through and between the seal proximal end and the seal distal end, and a tapered distal surface 116 disposed around the seal passage at the seal distal end. Seal body 104 also defines one or more grooves or channels to receive projection(s) 50 of cannula 14. As shown in FIG. 4B, body 104 defines distinct J-slots 120 on a peripheral surface of the body, with each J-slot configured to receive one of projections 50 of cannula 14. In the depicted configuration, each J-slot 120 includes a longitudinal portion 124 extending generally in the longitudinal direction, and a circumferential portion 128 extending in a generally circumferential direction around a peripheral portion of the seal body. In operation, seal distal end 112 is inserted into recess 42 of cannula 14 with projections 50 each aligned with corresponding one of longitudinal portions 124 until the projections reach the circumferential portions 128 of the J-slots 120, at which point the seal body (104) is rotated to position each projection 50 in the end of the corresponding circumferential portion 128 that is farthest from the corresponding longitudinal portion 124. In the depicted embodiment, one or more resilient annular seal members 132 are disposed on distal surface 116 and sized such that the seal members (132) are compressed to some degree when projections 14 of the cannula are disposed in circumferential portions 128 of the J-slots in seal body 104. Once compressed, these seal members 132 exert a force tending to urge the cannula proximal surface 34 and seal distal surface 116 apart, as well as ensure a degree of friction between the cannula proximal surface and the seal distal surface, to resist unintentional rotation of the seal body in a way that could otherwise unintentionally allow separation of the seal body from the cannula.

In the embodiment shown, seal assembly 100 also includes a resilient membrane 136 disposed at least around an interior periphery of the seal passage. Resilient membrane 136 is configured to cooperate with an elongated body of delivery tool 200 (as shown in FIG. 1) to substantially seal the seal passage (116) when the body of the apparatus extends through the resilient membrane and the seal passage. In this configuration, membrane 136 is attached directly to an interior surface 140 of seal body 104 that defines a proximal portion of seal passage 116, and extends radially inward to define a central membrane opening 144 that is aligned with a central longitudinal axis of the seal passage (116). In this configuration, opening 144 is circular to corresponding in shape to the circular cross-section of the elongated body of the delivery tool (200), and is sized with an interior diameter that is smaller than the exterior diameter of the corresponding portion of the elongated body, such that the resilient membrane stretches somewhat when the elongated body is inserted through opening 144 and the resilient membrane exerts a compressive force against the elongated body and provide a scaled interface therebetween.

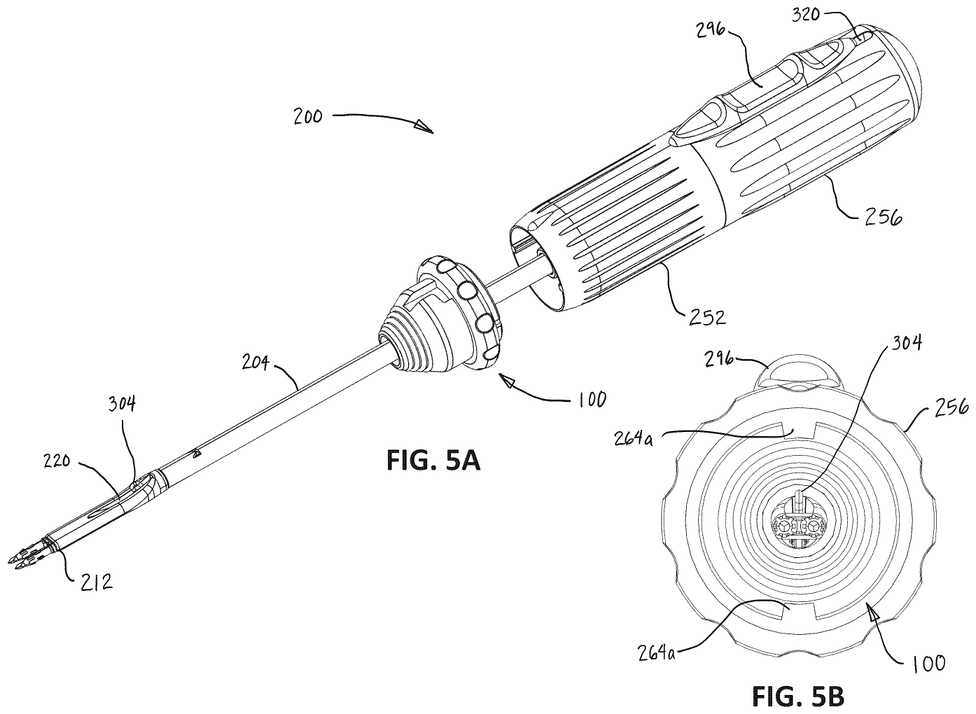

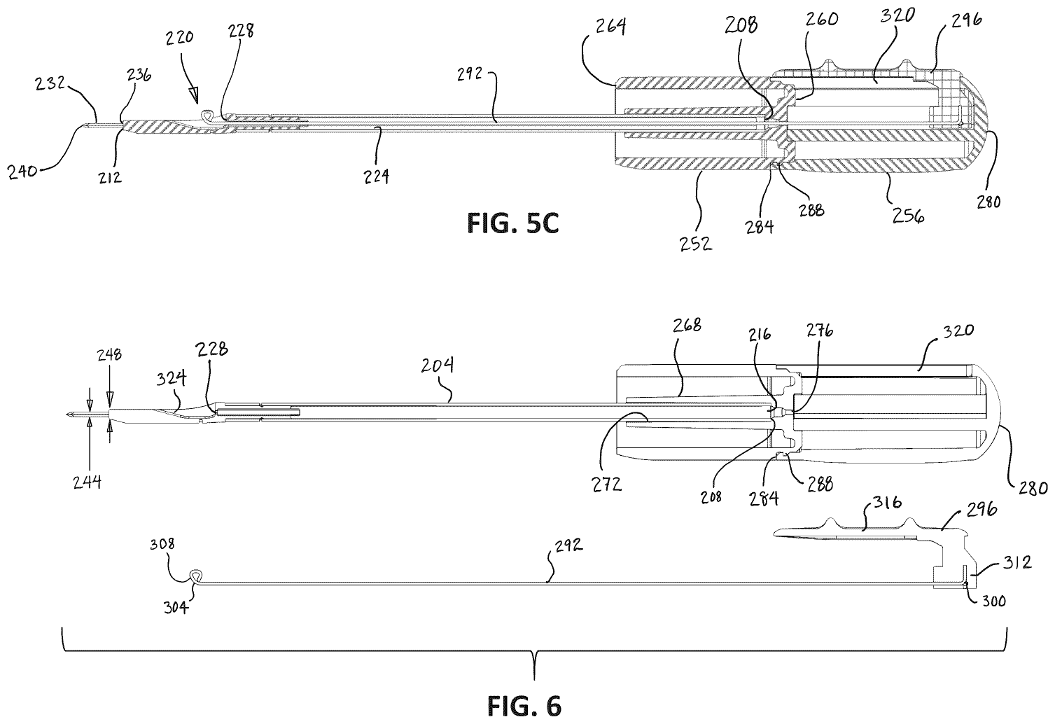

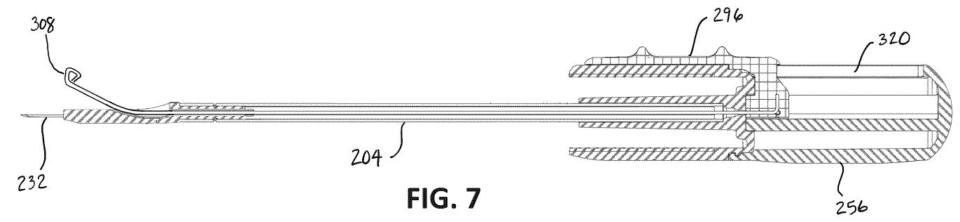

Referring now to FIGS. 5A-5C, 6, and 7; FIGS. 5A-5B show delivery tool 200 and seal assembly 100, with the delivery tool in a first configuration; FIG. 5C shows a cross-sectional view of the delivery tool in the first configuration; FIG. 6 shows a partially exploded side view of the delivery tool; and FIG. 7 shows a cross-sectional view of the delivery tool in a second configuration. As described in more detail below, the delivery tool is configured to deliver an implant and a fastener at the same time, such that the fastener can secure the implant to a portion of a patient (e.g., a bone or soft tissue) in a single motion. As shown, delivery tool 200 comprises an elongated body 204 extending from a body proximal end 208 to a body distal end 212. Body 204 defines a body proximal opening 216 through the body proximal end, a body recess 220 in a peripheral surface of the body at point that is offset from the body distal end (212), and a body passage 224 extending between the body proximal opening and a body distal opening 228 within the body recess. Delivery tool 200 also includes one or more spikes 232 (e.g., two spikes 232, in the depicted embodiment) each having a spike proximal end 236 coupled to the body distal end 212, and a spike distal end 240 extending from the body distal end. As shown, the spike proximal end has a transverse dimension 244 that is smaller than a transverse dimension 248 of the body distal end such that a shoulder is defined at the spike proximal end (and such that the shoulder is configured to abut a head of a fastener 600a received over the spike, as shown in FIG. 5A).

As shown, delivery tool 200 also includes a hub 252 and a handle 256. Hub 252 has a hub proximal end 260 and a hub distal end 264, and is configured to receive proximal end 208 of body 204 through the hub distal end. More particularly, hub 252 includes a portion 268 defining a recess 272 that is sized to receive proximal end 208 of body 204, which can be secured in recess 272 via a friction or interference fit, adhesive, and/or the like, such that the hub can be rotated to rotate the elongated body (204). Additionally, hub proximal end 260 defines a central opening 276 in fluid communication with and aligned with a central longitudinal axis of body passage 224. Handle 256 has a handle proximal end 280 and a handle distal end 284. As shown, handle distal end 284 is coupled to hub proximal end 208, for example, via one or more interlocking tabs and grooves or notches. By way of example, in the depicted configuration, hub proximal end 260 includes one or more tabs 288 that project radially outward, and handle distal end 284 defines an annular flange with one or more corresponding grooves that receive the tab(s) 288 to resist separation of the handle (256) from the hub (252) while still permitting the handle to rotate relative to the hub, at least within a defined angular range (e.g., less than any one of or between any two of: 30 degrees, 45 degrees, and/or 90 degrees). In the depicted configuration, such rotation of the handle (256) relative to the hub (252) also causes the pusher to rotate, such that when or as the distal end of the pusher (e.g., 292, 292a, 292b) is extended distally, the distal end of the pusher can move laterally to apply urge lateral portions of an implant (e.g., 500) toward the bone or other tissue to which the implant is to be secured.

Delivery tool 200 also includes an elongated pusher 292 and a slider 296. Pusher 292 has a pusher proximal end 300 and a pusher distal end 304, and slider 296 is coupled to pusher proximal end 300. Pusher distal end 304 includes a curved section 308 that is wider in the radial direction than an adjacent portion of the pusher, to resist snagging when the pusher slides against an implant as described below. In the depicted embodiment, pusher 292 is formed from an elongated wire, such as nitinol or stainless steel, with its distal end bent to define the curved section (308). In other embodiments, pusher 292 can be formed as a solid piece, in which the curved section is defined by machining or the like, or can be assembled from multiple pieces, such as a wire body and a paddle-shaped piece attached (e.g., welded) to the distal end of the wire to define the curved section.

As shown in FIG. 5C, pusher 292 is disposed in and extending through body passage 224 such that pusher distal end 304 is disposed beyond body distal opening 228. Slider 296 is coupled to pusher proximal end 300, and is configured to move longitudinally relative to the handle from a retracted first position (FIG. 5C) to a deployed position (FIG. 7) to move pusher distal end 304 longitudinally away from the body distal opening 228 and laterally outward from the body, as shown in FIG. 7. In the depicted configuration, slider 296 includes a hub portion 312 and a radial projection 316. Hub portion 312 aligns with the longitudinal axis of the body opening 224, and radial projection 316 extends radially outward through a slot 320 in handle 256 such that a user can push radial projection 316, such as with the user's thumb, to move slider 296 distally relative to handle 256 and extend pusher distal end 304 distally (from FIG. 5C vs. FIG. 7) and similarly shift radial portion 316 proximally to retract pusher distal end 304 (from FIG. 7 to FIG. 5C). As can be seen in FIGS. 5C, 6, and 7, body recess 220 defines a sloped surface 324 spaced from body distal opening 228 and positioned such that, when the slider is moved from the retracted position (FIG. 5C) to the deployed position (FIG. 7), first curved portion 308, and then an additional portion of the pusher, slide along sloped surface 324 to cause pusher distal end 304 to extend laterally or radially outward from body 204 as shown in FIG. 7.

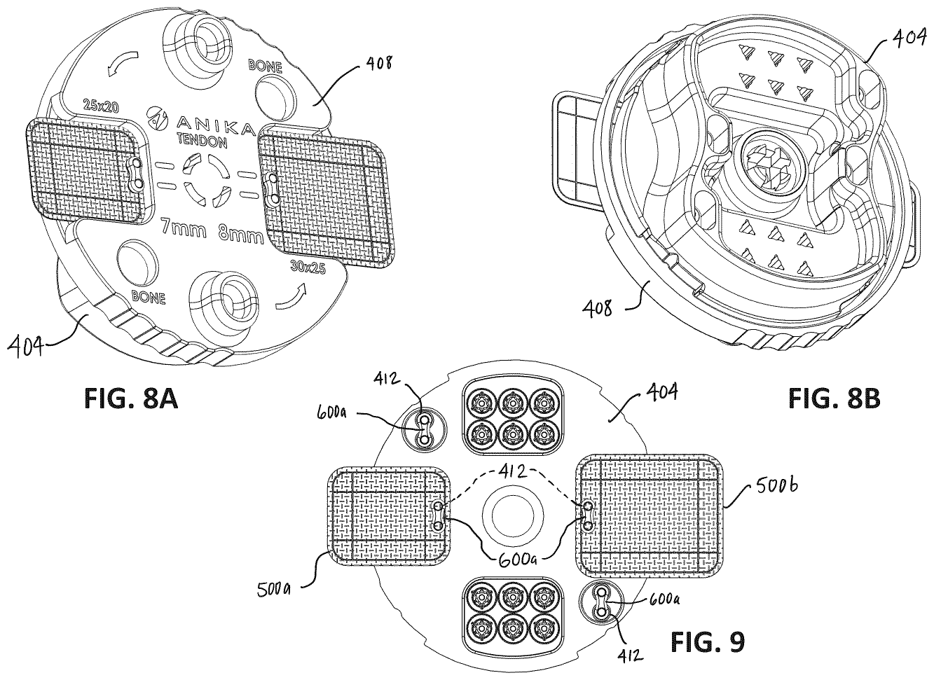

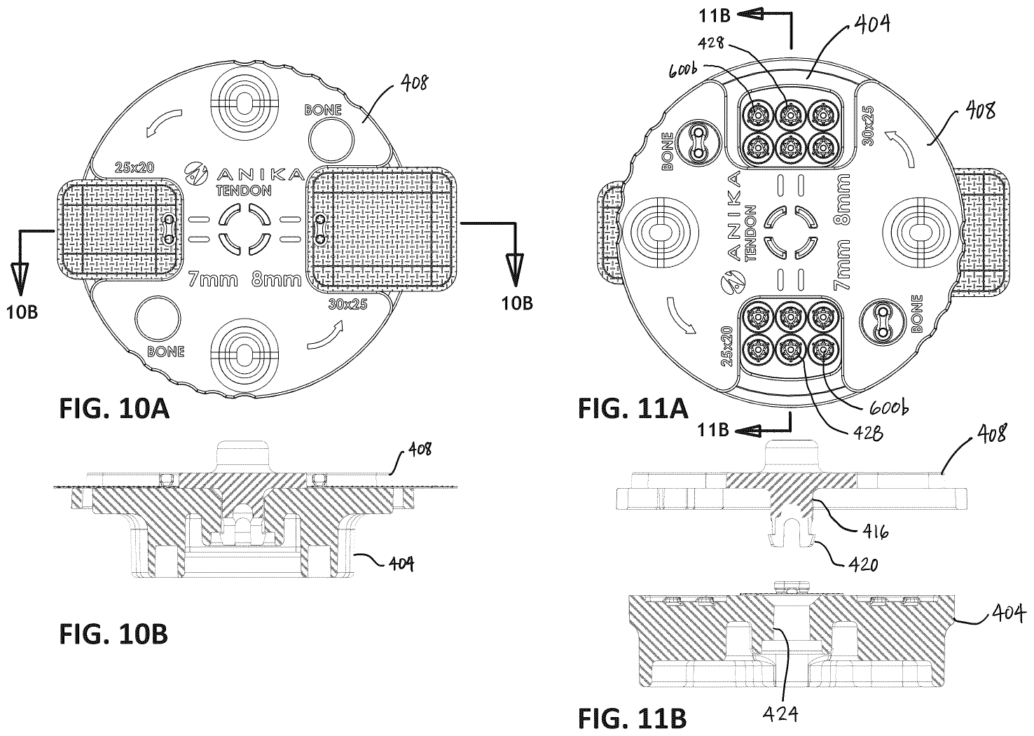

Referring now to FIGS. 8A-11B; FIGS. 8A and 8B depict upper and lower perspective views, respectively, of a first embodiment 400 of the present caddy systems shown pre-loaded with implants 500 and fasteners 600; FIG. 9 depicts a top view of a base portion 404 of caddy system 400; FIG. 10A depicts a top view of caddy system 400 with a lid portion 408 in a first configuration relative to base portion 404; FIG. 10B depicts a side cross-sectional view of caddy system 400 with the lid portion in the first configuration; FIG. 11A depicts a top view of caddy system 400 with lid portion 408 in a second configuration relative to base portion 404; and FIG. 11B depicts an exploded, side cross-sectional view of caddy system 400.

As shown generally, caddy system 400 incudes a base portion 404 and a lid portion 408. The base portion generally defines one or more first fastener recesses configured to receive a first type of fastener. For example, in the depicted embodiment, base portion 404 defines a plurality of first fastener recesses 412 configured to receive bone fasteners 600a (staple-type fasteners with two substantially parallel projections). For example, the lid portion 408 is configured to be rotatably coupled to the base portion such that the lid portion 408 may be rotated to selectively cover or expose the first fastener recesses (e.g., between FIG. 10A and FIG. 11A). In some embodiments, a recess is disposed at least partially in the lid portion or at least partially in the base portion, wherein the recess is configured to receive an implant. In some embodiments, the lid portion 408 is fixedly coupled with the base portion 404. In some embodiments, the lid portion 408 is detachably coupled from the base portion 404. For example, in the depicted embodiment, lid portion 408 includes a central shaft 416 extending from a lower side of the lid, and shaft 416 includes resilient barbs 420 such that the shaft can be inserted through a corresponding central opening 424 in the base. During insertion, barbs 420 are deflected radially inward and, once the shaft is fully inserted through opening 424, barbs 420 move radially outward to their relaxed state and thereby resist removal of the lid portion from the base portion (at least unless and until barbs 424 are subsequently deflected radially inward again).

In the embodiment shown, a first implant 500a is disposed over a first one of the first fastener recesses (412) and a bone fastener 600a extends through the first implant and into the corresponding first fastener recess, both to preload the first implant with a bone fastener and to resist movement of the first implant relative to base portion 404. In this configuration, spikes 232 of delivery tool 200 can be inserted into bone fastener 600a in first implant 500a to remove both of the bone fastener and the first implant and then simultaneously deliver that implant and fastener to a site in a patient and insert the bone fastener into a bone of a patient to secure the implant relative to that bone. In the depicted embodiment, a second implant 500b is also disposed over a second one of the first fastener recesses (412) and a second bone fastener 600a similarly extends through the second implant and into the corresponding first fastener recess. As shown, each of first and second implants are flexible sheet-like (e.g., fibrous) implants, and second implant 500b is larger than first implant 500a (e.g., 30 mm×25 mm versus 25 mm×20 mm). In other embodiments, the first and second implants may be the same size. In some embodiments, the upper side of base portion 404 also includes one or more implant recesses each overlapping at least one of the first fastener recesses 412.

Once the implant is initially secured to the bone, lid portion 408 can be rotated relative to the base portion 404 (e.g., from FIG. 10A to FIG. 11A) to expose additional bone fasteners 600a and soft tissue fasteners 600b. For example, base portion 404 also includes additional first fastener recesses 412 and corresponding bone fasteners 600a that may be used to provide additional securement of one or both of the implants to the corresponding bone(s). For example, after the initial bone fastener 600a is inserted to initially secure an implant to a bone, delivery tool 200 may be withdrawn and inserted through one of the additional bone fasteners in base portion 404, and the bone fastener can then be withdrawn from the base portion and inserted through the initially secured implant to further secure the implant to the bone. By way of further example, base portion 404 also includes a plurality of second fastener recesses 428 configured to receive soft-tissue fasteners 600b. As shown, soft tissue fasteners 600b are dart-like fasteners (with a single projection) that can be inserted with a different delivery tool having a single spike at its distal end (in other embodiments, the soft-tissue fasteners may take a staple-like configuration similar to that of bone fasteners 600a, such that the bone fasteners and soft-tissue fasteners can both be delivered with the same delivery tool). As described above for bone fasteners, a single-spike tool can be inserted through one of soft-tissue fasteners 600b and the fastener removed from base portion 404 and inserted through a corresponding implant into soft tissue of a patient. This can then be repeated until the implant is sufficiently secured relative to the soft tissue. As one example, for repair of the supraspinatus tendon, a first portion of the implant can first be secured to the humeral head via one or more bone fasteners 600a, after which a second portion of the implant can be secured to the tendon via one or more soft-tissue fasteners 600b.

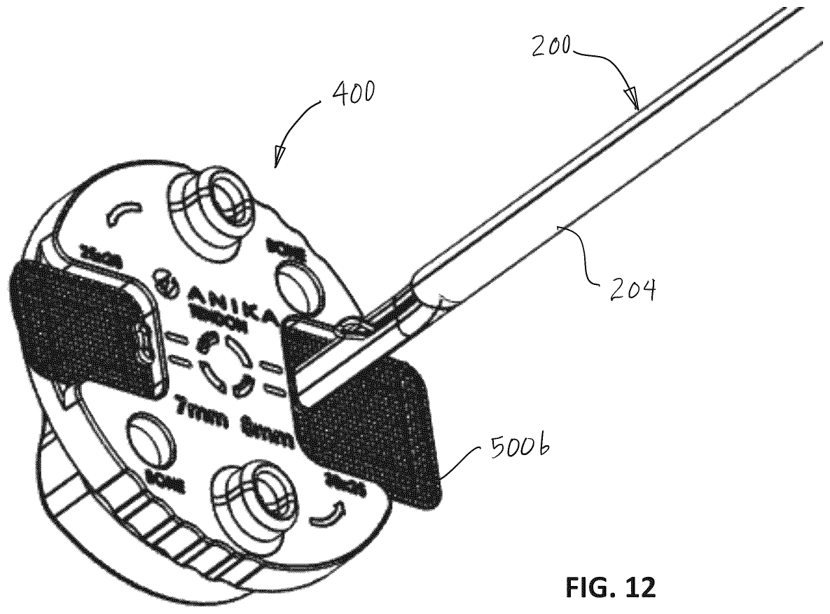

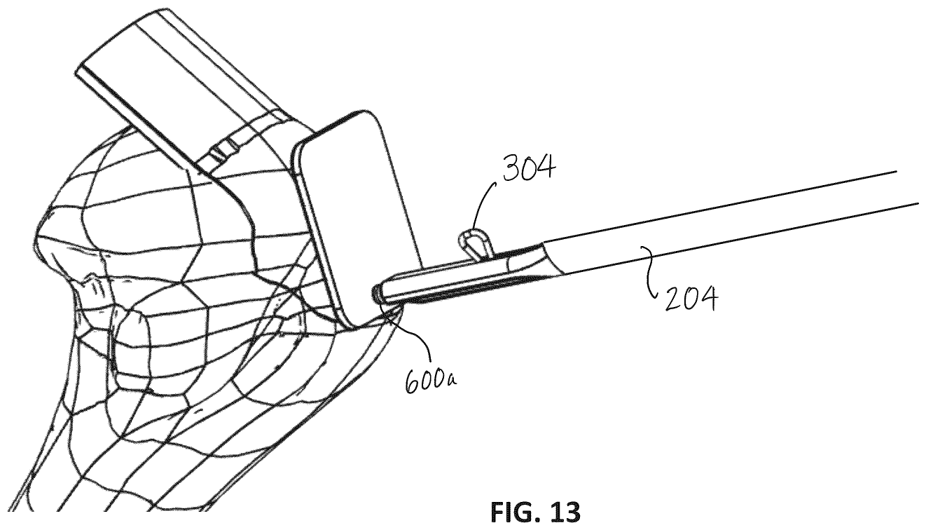

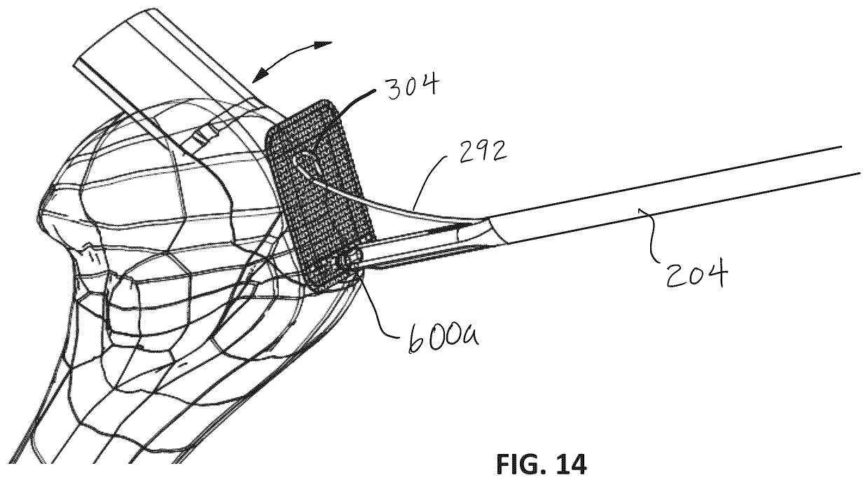

FIGS. 12-14 depict an example of a method of deploying an implant and fastener from caddy system 400 to a bone of a patient. As shown in FIG. 12, delivery tool 200 (with seal assembly 100 disposed around body 204 as shown in FIG. 1) is aligned with bone fastener 600a corresponding to implant 500b, and spikes 232 of the delivery tool inserted into the bone fastener. Implant 500b and the corresponding bone fastener are then removed from caddy system 400 and inserted through a cannula 14 (not shown in FIG. 13 for clarity) to deliver the implant and bone fastener to a position over a patient's humeral head. Typically before insertion of the bone fastener into the humeral head, the distal end of seal assembly 100 (not shown in FIG. 13 for clarity) is aligned with and inserted into recess 42 on the proximal end of cannula 14, and the seal assembly and cannula are secured together to seal the cannula so the region around the patient's humeral head can be filled with liquid such as saline to create space and improve visibility (typically via camera inserted through a separate port or incision). Once the seal assembly is secured to the cannula, and the region is optionally filled with liquid, the delivery tool can be manipulated to steer the implant and bone fastener to a desired location on the humeral head, and then to insert the bone fastener into the humeral head to secure a corresponding portion of the implant relative to the bone. Once the bone fastener is inserted, and as shown in FIG. 14, slider 296 of the delivery tool can be moved from its retracted position (FIG. 5C) to its extended position (FIG. 7 and FIG. 13) to cause distal end 304 of pusher 292 to move distally and radially outward relative to body 204. In doing so, curved portion 308 of pusher 292 slides along implant 500b to urge the offset portion of the implant (which is offset in the longitudinal dimension of the implant from fastener 600a) toward the tendon to facilitate proper placement of the implant relative to the tendon. Additionally, handle 256 of the delivery tool can be rotated relative to hub 252 to cause distal end 304 of pusher 292 to sweep clockwise and/or counterclockwise (as indicated by arrow 328) to urge lateral extends of the offset portion of the implant toward the tendon. Thereafter, seal assembly 100 can be disconnected from the cannula (e.g., after depressurizing the region around the humeral head) and delivery tool 200 and seal assembly 100 can be removed from the cannula, after which additional tools may be inserted (and additional seal assemblies attached) to deliver one or more soft-tissue fasteners (and/or to install sutures) to secure the offset portion of the implant to the tendon.

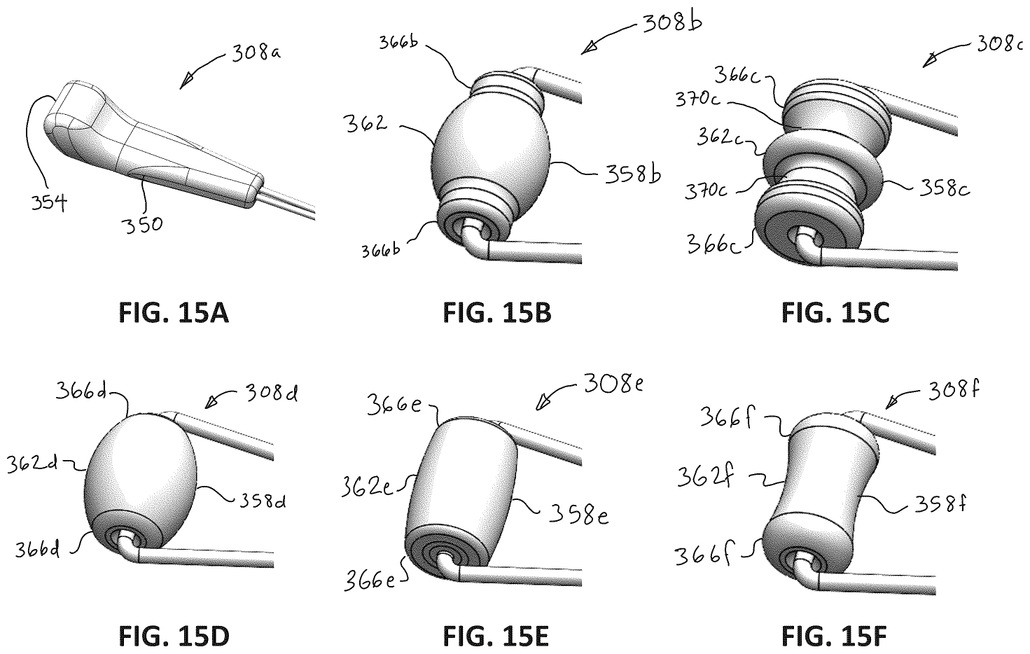

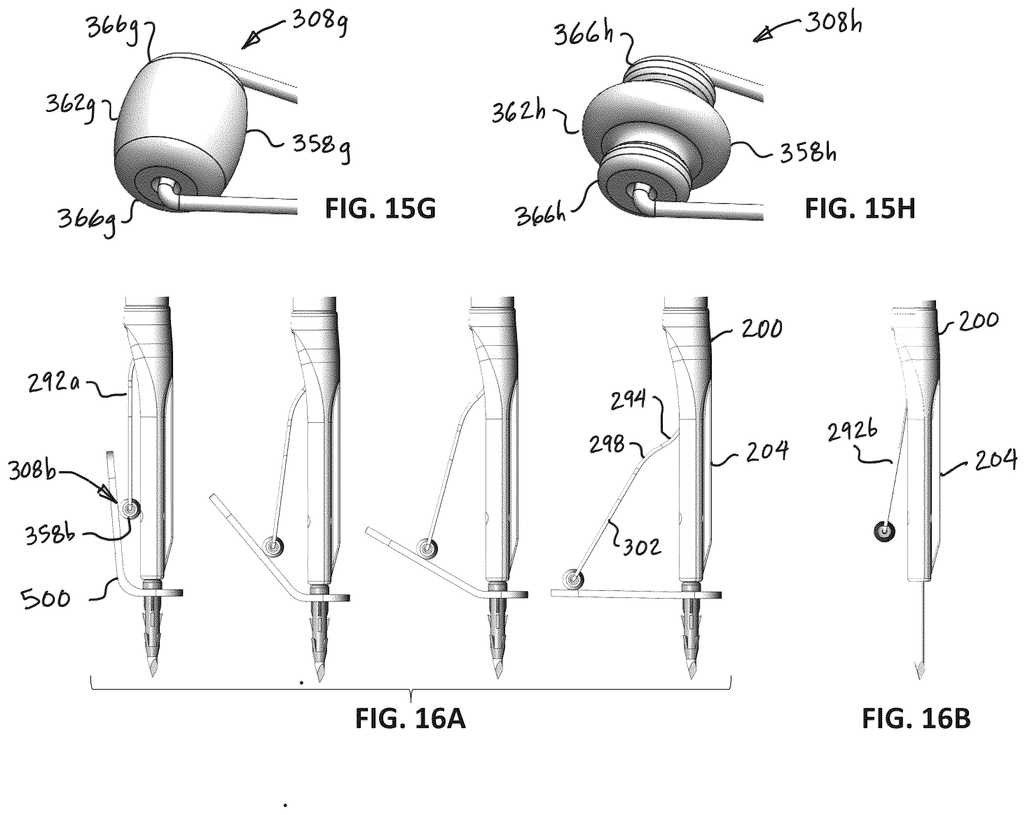

FIGS. 15A-15H depict perspective views of various configurations of a distal end of a pusher of the delivery tool 292. FIG. 15A depicts a first variation 308a of a distal end that includes an enlarged (relative to the wire or body of the pusher) club or foot 350 that is overmolded or otherwise coupled to the body or wire of the pusher). Club or foot 350 includes a curved distal surface 354 configured to slide relative to the implant to deploy the implant (e.g., FIG. 13 to FIG. 14).

FIGS. 15B-15H depict variations of distal end 308 that include rollers of various shapes configured to facilitate displacement of the distal end relative to an implant to deploy the implant (e.g., FIG. 16A). FIG. 15B depicts a first variation 308b of a distal end that includes a roller 358b configured to rotate around a portion of the wire or body of the pusher that defines a rotational axis that is substantially perpendicular to the direction of movement of the pusher during deployment of an implant (e.g., FIG. 16A). In other embodiments, roller 358b may be configured with an interference fit such that the roller may not rotate relative to the pusher during deployment of an implant; in such embodiments, however, the curved outer surface of the roller still facilitates movement of the pusher relative to the implant because of the curvature of that surface.

FIG. 15C depicts a further variation 308c of a distal end that includes a roller 358c. Roller 358c is similar to roller 358b, with the exception that roller 358c includes a relatively narrow medial section 362c that is separated by grooves 370c from lateral sections 366c, which have a common outer diameter that is the same as or slightly smaller (e.g., smaller by 10% or less) than the diameter of medial section 362c.

FIG. 15D depicts a further variation 308d of a distal end that includes a roller 358d. Roller 358d is similar to roller 358b, with the exception that roller 358d includes a convex, curved medial section 362d that spans substantially all of the width of the roller, and the maximum outer diameter of medial section 362d is between 20% and 80% (e.g., 40% to 50%) larger than the outer diameters of lateral sections or edges 366d.

FIG. 15E depicts a further variation 308e of a distal end that includes a roller 358c. Roller 358e is similar to roller 358d, with the exception that the outer diameter of medial section 362e is the same as or slightly larger (e.g., larger by 10% or less) than the diameter of lateral sections or edges 366c.

FIG. 15F depicts a further variation 308f of a distal end that includes a roller 358f. Roller 358f is similar to roller 358e, with the exception that the outer diameter of medial section 362c is slightly smaller (e.g., smaller by 10% or less) than the diameter of lateral sections or edges 366f, such that roller 308f defines a concave outer surface.

FIG. 15G depicts a further variation 308g of a distal end that includes a roller 358g. Roller 358g is similar to roller 358e, with the exception that the lateral sections 366g are each defined by a larger radius to define a more-rounded peripheral edge at either lateral end of the roller.

FIG. 15H depicts a further variation 308h of a distal end that includes a roller 358h. Roller 358h is similar to roller 358c, with the exception that the outer diameter of medial section 362h is between 20% and 80% (e.g., 40% to 50%) larger than the outer diameters of lateral sections 366h and medial section 362h is proportionally wider than medial section 362c.

FIG. 16A is a sequence of side views of a distal end of the delivery tool 200 with a further embodiment of the pusher 292a, illustrating the deployment of an implant (from left to right in illustrated sequence). As slide 296 (FIG. 5A) is advanced distally, distal end 308b of pusher 292 advances distally and radially away from elongated body 204. In the depicted embodiment, pusher 292a includes a wirelike body comprising a resilient material (e.g., stainless steel, nitinol or other shape-memory alloy, or the like) that is pre-shaped to assume a desired configuration upon extension from the interior of the elongated body (within which the shape of the pusher is constrained). As shown in the rightmost portion of FIG. 16A, pusher 292a includes a first curve 294 curving outward relative to the axis of the proximal portion of pusher 292a, a second curve 298 curving back inward relative to the axis of the proximal portion of pusher 292a, and an axial distal portion 302 extending from second curve 298 to distal end 308b. In this configuration, the tangent at the distal end of first curve 294 is angled at an angle of between 40 and 60 degrees (e.g., 45 degrees) relative to the axis of the proximal portion of pusher 292a, and second curve 298 reverses direction and curves back to a distal end tangent that is disposed at an angle of between 20 and 40 degrees relative to the axis of the proximal portion of pusher 292a. As a result, distal portion 302 of pusher 292a is disposed at an angle of between 20 and 40 degrees (e.g., 30 degrees) relative to the axis of the proximal portion of pusher 292a, and roller 358b is spaced from the body distal end 212 by a distance that spans a majority of a corresponding dimension of implant 500 to facilitate deployment of the implant, as shown. In the depicted examples, pusher 292a includes a wire extending through a central passage of the roller and having two similar portions extending to wire ends 300 within hub portion 312 of slider 296 (e.g., along a similar path as is shown in FIG. 6). In some examples, such as the one shown, pusher 292a comprises a 316 LVM Stainless Steel wire, for example, with a diameter of 0.021 mm or smaller.

Other configurations of the pusher may include any number of curves or bends to facilitate deployment of an implant. For example, FIG. 16B depicts a side view of a distal end of the delivery tool 200 with a further embodiment of the pusher 292b, which is substantially similar to pusher 292a with the exception that the wirelike body of pusher 292b includes a relaxed shape that is substantially linear (e.g., similar to pusher 292 in FIG. 6), as opposed to including pre-defined curves or angles. In this configuration, as pusher 292b is moved longitudinally relative to body 204 in a distal direction, sloped surface 324 will urge the distal end of pusher 292b laterally outward relative to body 204 and, as the roller (e.g., 358b) contacts tissue such as bone, the roller will roll against the tissue to facilitate the further lateral extension of pusher 292b. In this way, the roller may in some configurations be extended laterally outward until the roller is spaced from the body distal end 212 by a distance that spans a majority of a corresponding dimension of implant 500 to facilitate deployment of the implant, as shown.

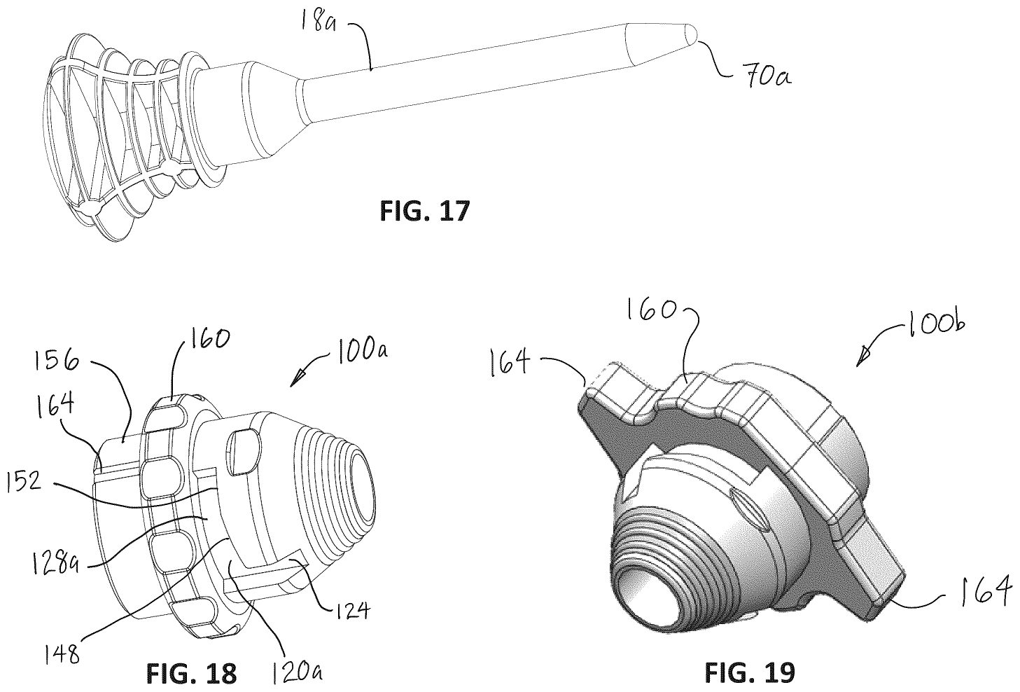

FIG. 17 is a perspective view of a further embodiment 18a of the obturator of FIGS. 1 and 2. Obturator 18a is substantially similar to obturator 18 of FIGS. 1 and 2, with the primary exception that obturator 18a has an obturator distal end 70a that conically tapers to a rounded end (as opposed to planar sides converging to a point).

FIG. 18 is a distal perspective view of a further embodiment 100a of the seal assembly of FIGS. 1 and 4A-4B. Seal assembly 100a is substantially similar to seal assembly 100, with the primary exceptions that seal assembly 100a includes modified J-slots 120a and an elongated seal body 104a. As shown in FIG. 18, each of J-slots 120a includes a longitudinal portion 124 extending generally in the longitudinal direction, and a circumferential portion 128a extending in a generally circumferential direction around a peripheral portion of the seal body. Circumferential portion 128a includes a first segment 148 and a second segment 152. First segment 148 includes a distal surface that extends helically around the perimeter of the seal body such that a line tangential to the curve of distal surface is angled relative to central, longitudinal axis of seal body 104a by an angle of between 80 degrees and 90 degrees (e.g., between 83 degrees and 87 degrees). In this embodiment, first segment 148 has a length in the circumferential direction that is at least 200% of a width (in the same direction) of longitudinal portion 124. Second segment 152 is configured such that its distal surface is substantially perpendicular to the central, longitudinal axis of seal body 104a. In the embodiment of FIG. 16, seal body 104a is also elongated (relative to seal body 104 of FIGS. 4A-4B) such that a proximal portion 156 extends from a knurled flange 160 to proximal end 108a by a greater length than that of seal body 104. Proximal portion 156 is sized to be received in hub distal end 264 of hub 252 (FIGS. 5B-5C), and proximal portion 156 also defines a pair of grooves 164 configured to receive ridges 264a (FIG. 5B) of the hub to resist relative rotation between seal body 104a and the hub when proximal portion 156 of the seal body is received in distal end 264 of the hub. In this configuration, the relatively greater length (and shallower angle) of first segment 148 allows for a more-gradual compression of seal members 132, reducing the amount of torque necessary to rotate seal assembly 100 relative to cannula 14; and the addition of second segment 152, which is perpendicular to the longitudinal axis of seal body 104, reduces the chance of counter-rotation of seal assembly 100 relative to cannula 14 during use (and potential loss of seal therebetween).

FIG. 19 is a distal perspective view of a further embodiment 100b of the seal assembly of FIGS. 1, 4A-4B, and 16. Seal 100b is similar to seal 100a, with the primary exception that seal 100b includes a pair of protruding handles 168 extending outward from the circumferential surface of knurled flange 160 to facilitate the application of a greater torque by a user (e.g., during single-handed operation).

FIG. 20 is a distal perspective view of a further embodiment 14a of the cannula of FIGS. 1, 2, and 3. Cannula 14a is similar to cannula 14, with the primary exception that cannula 14a defines a notch 24 at distal end 22. Notch 24 is configured to be aligned with distal end 308, 308a, 308b, etc. to create additional space for, and thereby facilitate deployment of, the implant via extension of the distal end of the pusher (e.g., as shown in FIG. 16). In this embodiment, cannula 14a also includes a visual and/or physical indicator 28 that is circumferentially aligned with notch 24 to enable a user (e.g., surgeon) to align slider 296, and thereby the distal end of the pusher, with notch 24. In the depicted embodiment, indicator 28 is a divot or impression that can be felt by a user without requiring visual confirmation; in other variations, indicator may be a protrusion, may be a painted indicator with minimal physical variation from the surrounding surface, or may be any other feature or combination of features by which a user can perceive the alignment of notch 24 while distal end 22 of the cannula is disposed in a patient.

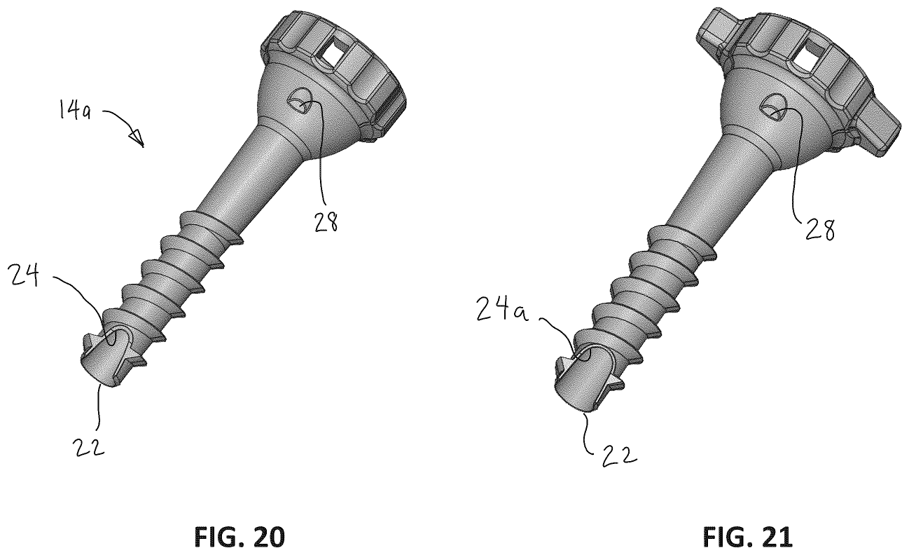

FIG. 21 is a distal perspective view of a further embodiment 14b of the cannula of FIGS. 1-3 and 18. Cannula 14b is similar to cannula 14a, with the primary exception that cannula 14b includes a pair of protruding handles 40 extending outward from the knurled circumferential surface of head or hub 38 to facilitate the application of a greater torque by a user (e.g., during single-handed operation).

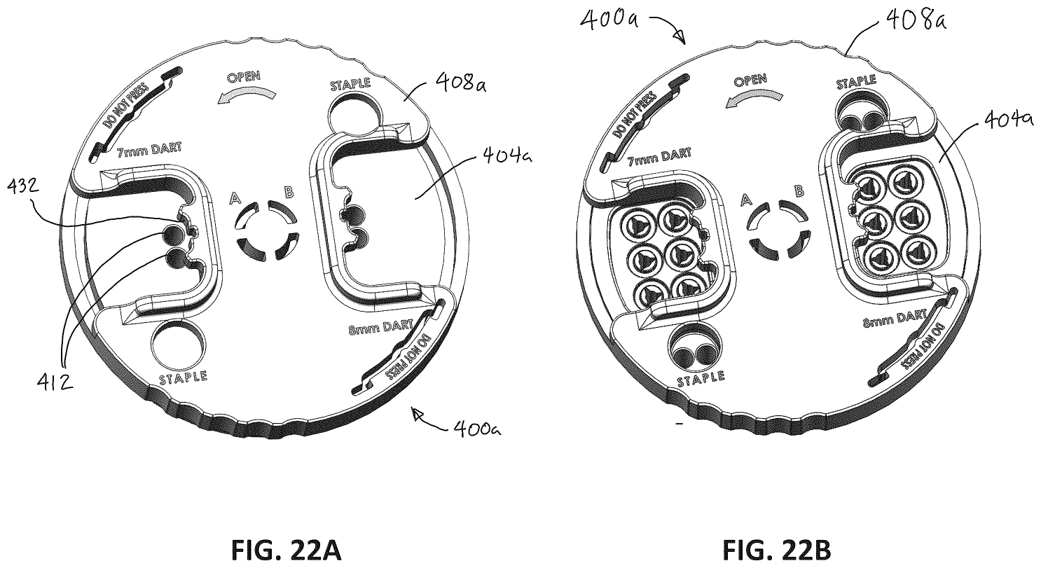

FIGS. 22A and 22B are upper perspective views of a further embodiment 400a of the present caddy systems. In FIG. 22A, lid portion 408a is in a first rotational position relative to base portion 404a in which the implants regions are accessible. In FIG. 22B, lid portion 408a is in a second rotational position relative to base portion 404a (rotated 90 degrees relative to the first rotational position of FIG. 22A) in which the implant regions are not accessible but the tendon (soft tissue) fasteners and additional bone fasteners are accessible. Caddy system 400a is substantially similar to caddy system 400, with the primary exception that lid portion 408a defines a tool guide having a lateral surface 432 disposed above (and spaced apart from) first fastener openings 412, with surface 432 configured to contact a portion of an elongated body 204 of tool 200, as the tool is inserted through a bone fastener (e.g., 600a) in or into first fastener recesses 412, to encourage alignment of the fastener (e.g., 600a) with first fastener recesses 412 and/or alignment of spikes 232 of the tool with the corresponding passages in the fastener. In the embodiment shown, surface 432 is shaped to contour to an exterior shape of the elongated body (204) of the tool (200). In other embodiments, surface 432 may include one or more bumps or peaks to facilitate centering or alignment of tools of different cross-sectional shapes.

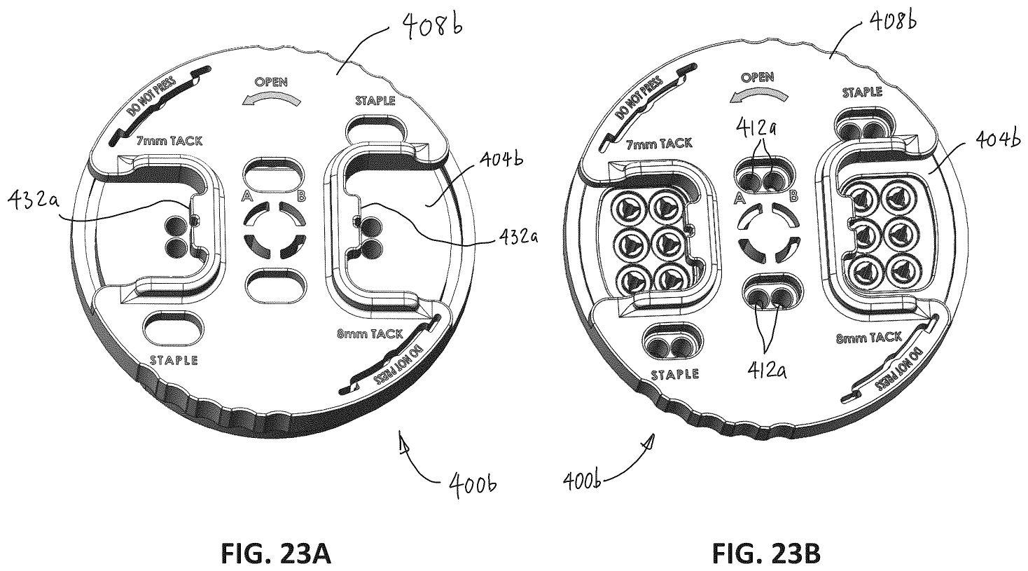

FIGS. 23A and 23B are upper perspective views of a further embodiment 400b of the present caddy systems. In FIG. 23A, lid portion 408b is in a first rotational position relative to base portion 404b in which the implants regions are accessible. In FIG. 23B, lid 408b is in a second rotational position relative to base portion 404b (rotated 90 degrees relative to the first rotational position of FIG. 23A) in which the implant regions are not accessible but the tendon (soft tissue) fasteners and additional bone fasteners are accessible. Caddy system 400b is substantially similar to caddy system 400a, with the primary exception caddy system 400b includes storage for an additional pair of bone fasteners and tool guides with surfaces 432a having a different shape. First, base portion 408b defines two additional first fastener recesses 412a, and lid portion 408b defines two additional corresponding openings to permit access to the bone fasteners in first fastener recesses 412a when lid 408b is in the second rotational position (FIG. 23B) relative to base portion 408b. Second, tool guide lateral surfaces 432a are each flat with a medial alignment notch rather than being contoured to a curved outer surface of a tool (e.g., 200). In this configuration, surfaces 432a are offset radially from the center of lid portion 408b to space the corresponding fasteners further from the edge of the corresponding implants; however, in other embodiments, surfaces 432 and 432a can be disposed at any position to achieve desired placement relative to the corresponding implants.

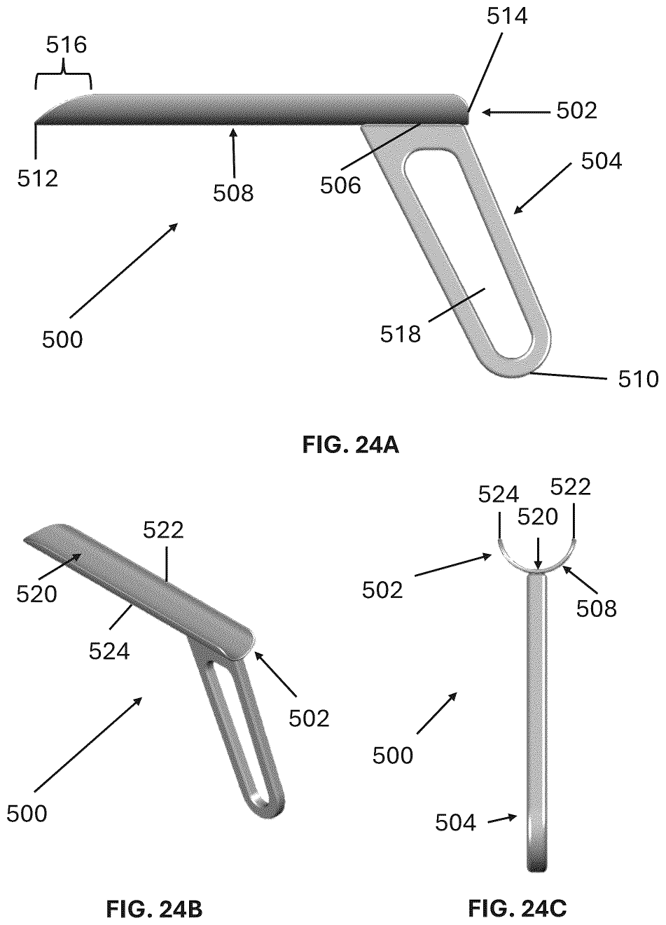

FIGS. 24A-24C illustrate different views of a guide cannula 500. In some embodiments, the guide cannula 500 comprises a slide portion 502 and a handle portion 504. In some embodiments, the guide cannula 500 is configured to be at least partially disposed in the cannula 14. For example, the outer dimensions of the slide portion 502 may be smaller than the inner diameter of the cannula 14 so as to be at least partially disposed within the cannula passage 30 and/or the cannula proximal surface 34. In some embodiments, the guide cannula 500 is configured to be at least partially inserted into the cannula 14 together with the delivery tool 200, such that the cannula guide at least partially encircles the delivery tool 200. In some embodiments, the guide cannula 500 is configured to be inserted at least partially within the cannula 14, so as to protect the cannula 14 from being punctured by the spike distal end 240 of the delivery tool 200. In some embodiments, the guide cannula 500 comprises a material more resistant to being punctured than the material of the cannula 14.

In some embodiments, the guide cannula 500 is configured to be at least partially disposed in any suitable commercially cannula. For example, the cannula guide 500 may be disposed in a flexible cannula. In some embodiments, the cannula guide 500 is substantially rigid and configured to provide a shape to a flexible cannula so as to provide space for the delivery tool 200 to pass therethrough. In some embodiments, the cannula guide 500 is configured to prevent puncture of the flexible cannula

As shown in FIG. 24A, in some embodiments, the handle portion 504 extends from a first end 506 which interfaces with the outer surface 508 of the slide portion 502 to a second end 510. In some embodiments, the handle portion 504 may extend at a non-zero angle relative to the longitudinal axis of the slide portion 502. In some embodiments, the handle portion 504 extends at a 45 degrees angle relative to the longitudinal axis of the slide portion 502. In some embodiments, the handle portion extends at a 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, or 125-130 degrees angle relative to the longitudinal axis of the slide portion 502.

In some embodiments, the slide portion 502 extends from a distal end 512 to a proximal end 514 along the longitudinal axis of the slide portion 502. In some embodiments, the slide portion 502 extends along the longitudinal axis of the slide portion 502 for a length greater than the length of a cannula. In some embodiments, the slide portion 502 extends along the longitudinal axis of the slide portion 502 for a length greater than or equal to the length of a cannula combined with the length of handle portion 504 coupled with the slide portion 502. In some embodiments, a tapered region 516 of the slide portion 502 extends from the distal end 512 to a distance spaced apart from the proximal end 514. In some embodiments, the tapered region 516 extends along less than 25% of the length of the slide portion 502. In some embodiments, the tapered region 516 extends along less than 50% of the length of the slide portion 502. In some embodiments, the tapered portion 510 tapers at an angle between 70-20 degrees from a location between the distal end 512 and the proximal end 514 to the distal end 512. In some embodiments, the tapered portion 510 tapers at an angle 45 degrees from a location between the distal end 512 and the proximal end 514 to the distal end 512.

In some embodiments, the handle portion 504 is configured to fixedly couple with the slide portion 502. In some embodiments, the handle portion 504 is configured to releasably couple with the slide portion 502. In some embodiments, the handle portion 504 extends from the proximal end 514 of the slide portion 502 along the longitudinal axis of the slide portion 502 to a desired location between the distal end 512 and the proximal end 514 of the slide portion 502. For example, the handle portion 504 may extend outwardly from and for a distance along the longitudinal axis of the slide portion 502 less than 50% of the length of the slide portion 502. For example, the handle portion 504 may extend outwardly from and for a distance along the longitudinal axis of the slide portion 502 less than 25% of the length of the slide portion 502.

In some embodiments, the handle portion 504 comprises a opening 518 passing through at least a portion of the handle portion. In some embodiments, the opening 518 has an outer dimension configured to permit a human hand to pass therethrough. In some embodiments, the opening 518 has an outer dimension configured to permit a human finger to pass therethrough. In some embodiments, the opening 518 has an outer dimension configured to permit one, two, three, or four fingers to pass therethrough. In some embodiments, the opening 518 has an oblong shape where the longitudinal axis of the opening 518 is coaxial with the longitudinal axis of the handle portion 504.

FIG. 24B illustrates a perspective view of the cannula guide 500 shown in FIG. 24A. In some embodiments, the slide portion 502 is curved such that the outer surface 508 has a convex curvature and an inner surface 520 has a convex curvature. In some embodiments, the cannula guide 500 has a truncated cylindrical shape. For example, the inner surface 520 may extend in a curved shape between a first edge 524 and a second edge 522 of the slide portion 502. In some embodiments, the first edge 522 and the second edge 524 extend parallel to the longitudinal axis of the slide portion 502.

FIG. 24C illustrates a distal view of the cannula guide 500 shown in FIG. 24A. In some embodiments, the inner surface 520 forms a concave curvature between the first edge 522 and the second edge 524. In some embodiments, the concave curvature of the inner surface 520 is substantially spherical. In some embodiments, the inner surface 520 forms a sector having a central angle between 30-40, 40-50, 50-60, 60-70, 60-80, 80-90, 90-100, 100-110, 110-120, 120-30, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, or 350-360 degrees. In some embodiments, the inner surface 520 forms a sector having a central angle between 160-180 or 180-200 degrees. In some embodiments, the inner surface 520 forms a sector having a central angle of 180 degrees.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An apparatus for simultaneous delivery of a fastener and an implant, the apparatus comprising:
an elongated body extending from a body proximal end to a body distal end, the body defining a body proximal opening through the body proximal end, a body recess in a peripheral surface of the body at point that is offset from the body distal end, and a body passage extending between the body proximal opening and a body distal opening within the body recess;
one or more spikes each having a spike proximal end coupled to the body distal end, and a spike distal end extending from the body distal end, the spike proximal end having a transverse dimension that is smaller than a transverse dimension of the body distal end such that a shoulder is defined at the spike proximal end, the shoulder configured to abut a head of a fastener received over the spike;
a hub having a hub proximal end and a hub distal end, the hub configured to receive the proximal end of the body through the hub distal end;
a handle coupled to the hub proximal end;
an elongated pusher having a pusher proximal end and a pusher distal end, the pusher disposed in the body passage such that the pusher distal end is disposed beyond the body distal opening; and
a slider coupled to the pusher proximal end, the slider configured to move longitudinally relative to the handle from a retracted position to a deployed position to move the pusher distal end longitudinally away from the body distal opening and laterally outward from the body.

2. The apparatus of claim 1, where the one or more spikes comprise a pair of spikes, where each of the pair of spikes are parallel to and laterally spaced from the other of the pair of spikes.

3. The apparatus of claim 1, where the hub is coupled in fixed relation to the body, and the handle is rotatable relative to the hub to cause the pusher distal end to rotate relative to the body.

4. The apparatus of claim 3, where the apparatus is configured such that moving the slider longitudinally relative to the handle, or rotating the handle relative to the hub, will move the distal end of the pusher independent of the body distal end.

5. The apparatus of claim 1, where the body recess defines a sloped surface spaced from the body distal opening, and where the apparatus is configured such that, when the slider is moved from the retracted position to the deployed position, a flexible portion of the pusher contacts the sloped surface to cause the pusher distal end to extend laterally outward from the body.

6. The apparatus of claim 1, where the pusher comprises a roller coupled to the pusher distal end.

7. The apparatus of claim 1, further comprising:
a seal assembly comprising:
an annular seal body having a seal proximal end and a seal distal end, the seal body defining a seal passage extending through and between the seal proximal end and the seal distal end, and a tapered distal surface disposed around the seal passage at the seal distal end; and
a resilient membrane disposed at least around an interior periphery of the seal passage, the resilient membrane configured to cooperate with the body of the apparatus to substantially seal the seal passage when the body of the apparatus extends through the resilient membrane and the seal passage.

8. The apparatus of claim 7, where the elongated body of the apparatus is disposed through the resilient membrane of the seal and the seal passage with the seal distal end facing the body distal end.

9. A system comprising:
an apparatus for simultaneous delivery of a fastener and an implant, the apparatus comprising:
an elongated body extending from a body proximal end to a body distal end, the body defining a body proximal opening through the body proximal end, a body recess in a peripheral surface of the body at point that is offset from the body distal end, and a body passage extending between the body proximal opening and a body distal opening within the body recess;
one or more spikes each having a spike proximal end coupled to the body distal end, and a spike distal end extending from the body distal end, the spike proximal end having a transverse dimension that is smaller than a transverse dimension of the body distal end such that a shoulder is defined at the spike proximal end, the shoulder configured to abut a head of a fastener received over the spike;
a hub having a hub proximal end and a hub distal end, the hub configured to receive the proximal end of the body through the hub distal end;
a handle coupled to the hub proximal end;
an elongated pusher having a pusher proximal end and a pusher distal end, the pusher disposed in the body passage such that the pusher distal end is disposed beyond the body distal opening;
a slider coupled to the pusher proximal end, the slider configured to move longitudinally relative to the handle from a retracted position to a deployed position to move the pusher distal end longitudinally away from the body distal opening and laterally outward from the body;
a seal assembly comprising:
an annular seal body having a seal proximal end and a seal distal end, the seal body defining a seal passage extending through and between the seal proximal end and the seal distal end, and a tapered distal surface disposed around the seal passage at the seal distal end; and
a resilient membrane disposed at least around an interior periphery of the seal passage, the resilient membrane configured to cooperate with the body of the apparatus to substantially seal the seal passage when the body of the apparatus extends through the resilient membrane and the seal passage;
and
a cannula comprising;
a cannula distal end, a cannula proximal end configured to be coupled to the seal body, and a cannula passage extending through and between the cannula proximal end and the cannula distal end, the cannula further defining a cannula proximal surface tapered distally and inward toward the cannula passage, the cannula proximal surface configured to cooperate with the seal distal surface to provide a seal between the cannula proximal surface and the seal distal surface.

10. The system of claim 9, where an outer surface of the cannula includes one or more threads that are configured to engage soft tissue as the cannula is rotated.

11. The system of claim 9, where the cannula proximal end includes an enlarged portion that is configured to be engaged by a user to vary the depth and/or direction of the cannula.

12. The system of claim 9, where the cannula distal end defines a notch configured to permit a portion of the elongated pusher to deflect radially outward through the notch.

13. The system of claim 9, further comprising:
an obturator having an elongated body with a obturator proximal end and a tapered obturator distal end, the obturator body configured to extend through the cannula such that the obturator distal end extends distally beyond the cannula distal end to facilitate insertion of the cannula through soft tissue of a patient.

14. The system of claim 9, further comprising:
a guide cannula having a slide portion and a handle portion, wherein the slide portion is configured to be at least partially disposed in the cannula passageway.

15. The system of claim 14, wherein the slide portion has an outer surface having a convex curvature and an inner surface having a convex curvature the inner surface extending between a first edge and a second edge of the slide portion, wherein the inner surface forms a sector having a central angle between 160-180 or 180-200 degrees, preferably 180 degrees.

16. The system of claim 14, wherein the slide portion extends from a distal end to a proximal end, wherein the slide portion comprises a tapered region extending from the distal end to a distance spaced apart from the proximal end.

17. The system claim 14, wherein the slide portion has an outer dimension smaller than the dimensions of the cannula passageway and an inner surface having a dimension larger than the outer dimension of a distal end of the apparatus of claim 1, such that the guide cannula protects the cannula from being punctured by the one or more spikes of the apparatus.

18-36. (canceled)
